(19) United States
(12) Patent Application Publication
(10) Pub. No.: US 2024/0158470 A1
(43) Pub. Date: May 16, 2024

(54) SYSTEMS AND METHODS FOR THE PREPARATION OF ENRICHED SERUM

(71) Applicant: Arthrex, Inc., Naples, FL (US)

(72) Inventors: Christopher Bare, Naples, FL (US); Hannah McIntyre, Naples, FL (US); Matthew Wittock, Naples, FL (US); Andrea Matuska, Naples, FL (US); Robert Harrison, Naples, FL (US)

(73) Assignee: Arthrex, Inc., Naples, FL (US)

(21) Appl. No.: 18/549,454

(22) PCT Filed: Mar. 10, 2022
(86) PCT No.: PCT/US2022/019677

§ 371 Date: Sep. 7, 2023

Related U.S. Application Data
(60) Provisional application No. 63/159,166, filed on Mar. 10, 2021.

(51) Int. Cl. C07K 14/715; A61B 5/15; A61K 38/17

(52) U.S. Cl. CPC C07K 14/7155; A61B 5/150755; A61K 38/1793

(57) ABSTRACT

Systems and methods for preparing enriched biological samples, and methods of use thereof are described herein. The systems can include a containment device connected to a first insert to collect tissue fragments in the containment device or to a second insert to incubate tissue fragments with a blood sample in the containment device. The methods can include methods for preparing a cytokine or growth factor-enriched biological sample, such as IL-1 Ra-enriched serum, methods of treating inflammation, inflammatory joint, or rheumatoid arthritis, and methods of preventing inflammation of a joint following arthroscopic surgery.

### PRIORITY

This application claims the benefit of U.S. Ser. No. 63/159,166, filed on Mar. 10, 2021, which is incorporated by reference in its entirety.

### BACKGROUND

Inflammatory cytokine interleukin-1 (IL-1) plays a key role in accelerating tissue destruction and repair mechanisms. Interleukin-1 receptor antagonist (IL-1Ra) balances IL-1 concentrations to maintain healthy joints. During surgical procedures, arthroscopic debridement, and/or resection of soft tissue, cartilage, bone, cells, and other large particles, which naturally produce IL-1Ra and other substances such as IL-6, IL-10, VEGF, EFG, HGF, PDGF, IGF, TGF-β, and BMP-9, are liberated from their resident tissues. There is a need for devices and methods for collecting the resected tissue fragments and using them to prepare biological fluid containing, for example, enriched amounts of cytokines and growth factors such as IL-1Ra, IL-6, IL-10, VEGF, EFG, HGF, PDGF, BMP-9, IGF, TGF-β, and other factors.

### SUMMARY OF THE DISCLOSURE

Tissue fragments that are liberated during arthroscopic surgery can be used for preparing enriched biological fluid. Therefore, there is a need for improved devices and methods for collecting and processing tissue fragments liberated during arthroscopic procedures and using them to prepare enriched biological fluid. Such devices and methods have substantial application in, for example, orthopedics and treatment of joint inflammation.

An embodiment provides a system for preparing a serum comprising: a) a containment device; b) a cap that fits within the containment device comprising a seal, an aperture, and a connector that can be connected to a first insert and to a second insert; c) a first insert comprising: i) a first adaptor configured to allow a fluid to flow into the containment device through the first insert; ii) a second adaptor configured to allow a fluid to flow out of the containment device through the first insert; iii) a cylindrical filter connected to the second adaptor, configured to extend into the containment device; and iv) a connector that can be connected to the cap; d) a second insert comprising: i) a connector that can be connected to the cap, and ii) a first port that can comprise a first port cap.

The containment device can comprise a first opening at a proximal end to accommodate the cap, and a second opening at a distal end comprising a second port that can comprise a second port cap. The seal can be an O-ring. The seal can fully contact an inner wall or inner walls of the containment device. The filter can be configured to retain tissue fragments inside the containment device. The system can further comprise a device for resecting tissue fragments and fluid from a surgical site connected to the first adaptor. The device can be an endoscopic shaver, an endoscopic bone cutter, an endoscopic burr, or an endoscopic drill. The system can further comprise a device for collecting tissue fragments and fluid from a surgical site connected to the second adaptor. The device can be a suction wand. The containment device can comprise a clear material. The containment device can comprise graduated marks for measuring a volume of tissue fragments and fluid collected in the containment device. The first port, the second port, or both the first port and the second port can be slip tip, luer-slip or luer-lok style ports (e.g., a port having a friction-fit connector). The containment device can comprise activating, concentrating, or both activating and concentrating materials. The activating, concentrating, or both activating and concentrating materials can comprise glass beads or polyacrylamide beads.

Another embodiment provides a method of producing a cytokine-enriched biological sample, a growth factor-enriched biological sample, or both, with the systems described herein. The methods comprise connecting the first insert to the cap, wherein the cap is located within the containment device at the proximal end of the containment device; connecting the first adaptor of the first insert to a device for liberating fragments from a tissue in a fluid and connecting the second adaptor of the first insert to a suction or aspiration apparatus to collect tissue fragments in the containment device and on the filter; removing tissue fragments from the filter to pool tissue fragments in the containment device; removing the first insert and connecting the second insert of the system to the cap; centrifuging the system to separate the tissue fragments from the fluid and removing the fluid from the containment device; and adding a biological sample to the containment device and incubating the tissue fragments with the biological sample, thereby producing a cytokine-enriched biological sample, a growth factor-enriched biological sample, or both.

Another embodiment provides a method of producing an interleukin-1 receptor antagonist (IL-1Ra)-enriched biological sample with the system described herein comprising: a) connecting the first insert to the cap, wherein the cap is located within the containment device at the proximal end of the containment device; b) connecting the first adaptor of the first insert to a device for liberating fragments from a tissue in a fluid and connecting the second adaptor of the first insert to a suction or aspiration apparatus to collect tissue fragments in the containment device and on the filter; c) removing tissue fragments from the filter to pool tissue fragments in the containment device; d) removing the first insert and connecting the second insert of the system to the cap; e) centrifuging the system to separate the tissue fragments from the fluid and removing the fluid from the containment device; and f) adding a biological sample to the containment device and incubating the tissue fragments with the biological sample, thereby producing an IL-1Ra-enriched biological sample.

The containment device can comprise activating, concentrating, or both activating and concentrating materials. The activating concentrating or both activating and concentrating materials can comprise glass beads or polyacrylamide beads. Removing the fluid can comprise connecting a syringe to the first port of the second insert and collecting the fluid in the syringe. Adding the biological sample to the containment to the containment device can comprise injecting a biological sample in the first port of the second insert or in the second port of the containment device. The method can comprise centrifuging the system to separate the enriched biological sample into a readily available fraction. The biological sample can be blood, bone marrow, effusion fluid, synovial fluid, or an adipose tissue sample. The biological sample can be a blood sample and the enriched biological sample can be a cytokine-enriched biological sample or the growth factor-enriched biological sample such as an IL-1Ra enriched serum. The method can comprise further collecting the cytokine-enriched biological sample or the growth factor-enriched biological sample, wherein collecting the cytokine-enriched biological sample or the growth factor-enriched biological sample can comprise connecting a syringe to the first port of the second insert and collecting the cytokine-enriched biological sample or the growth factor-enriched biological sample in the syringe. Collecting the cytokine-enriched biological sample or the growth factor-enriched biological sample from the containment device in the syringe can actuate the cap down within the containment device toward the distal end of the containment device. Injecting the biological sample in the containment device with a syringe can actuate the cap up within the containment device toward the proximal end of the containment device. Incubating the tissue fragments with the biological sample can be for about 4 hours or more. The tissue can be soft tissue, bone, or cartilage. The soft tissue can be tendon, ligament, muscle, adipose, or fascia. The device for liberating cells can be an arthroscopic shaver, an arthroscopic bone cutter, an arthroscopic burr, or an arthroscopic drill. The suction or aspiration apparatus can be a suction wand. The blood sample can be a non-anticoagulated blood sample, a platelet-rich plasma (PRP) sample, or another concentrated blood fraction. Removing tissue fragments from the filter can comprise scraping tissue fragments from the filter and into the containment device.

The cytokine-enriched biological sample, the growth factor-enriched biological sample, or both are enriched with one or more of IL-1Ra, IL-6, IL-10, VEGF, EFG, HGF, PDGF, BMP-9, IGF, and TGF-β.

An additional embodiment provides a method of treating inflammation, inflammatory joint, or rheumatoid arthritis in a subject comprising administering to the subject an IL-1Ra-enriched biological sample produced by the method described herein.

The cytokine-enriched biological sample or the growth factor-enriched biological sample can be injected at an inflammation site, inflammatory joint, or rheumatoid arthritis site. The cytokine-enriched biological sample or the growth factor-enriched biological sample can be an IL-1Ra-enriched serum.

A further embodiment provides a method of preventing inflammation of a joint following an arthroscopic surgery in a subject comprising administering into the joint an cytokine-enriched biological sample or the growth factor-enriched biological sample produced by the methods described herein. The joint can be, for example, a knee, an elbow, a shoulder, a wrist, an ankle, or a hip.

### BRIEF DESCRIPTION OF THE DRAWINGS

The accompanying drawings are included to provide a further understanding of the methods and compositions of the disclosure, are incorporated in, and constitute a part of this specification. The drawings illustrate one or more embodiments of the disclosure, and together with the description serve to explain the concepts and operation of the disclosure.

FIGS. 1A-D illustrate a system for preparing a serum. FIG. 1A shows a containment device. FIG. 1B shows a first insert. FIG. 1C shows a second insert. FIG. 1D shows a cap.

FIG. 2 schematically illustrates how a fluid flows into and out of a containment device connected to a first insert.

FIG. 3 schematically illustrates how a second insert actuates into a containment device.

FIG. 4 schematically illustrates a containment device comprising blood and tissue fragments.

FIG. 5 schematically illustrates a prepared serum separated from the blood and tissue fragments in a containment device following centrifugation.

FIGS. 6A-B show schematic depictions of actual photographs of serums prepared according to embodiments of the present disclosure. FIG. 6A shows containment devices comprising bone (left) and cartilage (right) tissue fragments in blood after four hours of incubation, before centrifugation. FIG. 6B shows containment devices comprising serum prepared from bone (left) and cartilage (right) in blood after four hours of incubation and after centrifugation.

### DETAILED DESCRIPTION

Overview

Defects in articular cartilage can induce osteoarthritis by causing molecular changes in the synovial fluid. Inflammatory cytokine IL-1 has been established as an inducer of osteoarthritis, which plays a role in accelerating tissue destruction and repair mechanisms. In a healthy joint, IL-1 and interleukin-1 receptor antagonist (IL-1Ra) are in balanced concentrations. In cases of osteoarthritis, there is not sufficient IL-1Ra produced to block the destructive effects of the increased IL-1. The result is inflammation, joint pain, and eventually cartilage destruction. The present disclosure provides systems and methods to stimulate monocytes to produce regenerative and anti-inflammatory proteins such as IL-1Ra without the addition of drugs. The systems and methods can also enrich amounts of cytokines and growth factors such as IL-6 (interleukin 6), IL-10 (interleukin 10), VEGF (vascular endothelial growth factor), EFG (epidermal growth factor), HGF (human growth factor) PDGF (platelet-derived growth factor) BMP-9 ((bone morphogenetic protein, 9), IGF (insulin-like growth factor), TGF-β (transforming growth factor beta), and other factors from biological samples.

### Problem to be Solved

Healthy joints naturally produce balanced levels of cytokines and growth factors like IL-1 and IL-1Ra. During arthroscopic surgeries, tissue fragments from various origins can be generated. There is a need for improved devices and methods for collecting and processing tissue fragments that can naturally stimulate the production of, for example, growth factors and cytokines such as IL-1Ra, IL-6, IL-10, VEGF, EFG, HGF, PDGF, BMP-9, IGF, TGF-β, and other factors. The enriched biological compositions, such as enriched serum compositions, made using the devices can be used for, e.g., treating inflammation, inflammatory joint issues, or rheumatoid arthritis in a subject, and to prevent inflammation of a joint following an arthroscopic surgery in a subject.

System for Preparing a Serum

A system for preparing a serum can comprise a containment device (100), a cap (400), a first insert (200), and a second insert (300).

A containment device (100) can comprise a first opening (120) at a proximal end (130) to accommodate a cap (400), and a second opening (150) at a distal end (140) comprising a port (110) that can comprise a port cap. A containment device can comprise a clear material. A containment device can comprise graduated marks for measuring a volume of tissue fragments and fluid collected in the containment device.

A cap (400) can fit within a containment device (100). A cap can comprise a seal (410), an aperture that runs longitudinally through the cap, and a connector (420) that can be connected to a first insert (200) and to a second insert (300). A seal (410) can be an O-ring. A seal can fully contact an inner wall or inner walls of a containment device.

A first insert (200) can comprise two adaptors (210, 220). A first adaptor (220) can be configured to allow a fluid to flow into a containment device through the first insert (200) and a second adaptor (210) can be configured to allow a fluid to flow out of a containment device through the first insert. A first insert can comprise a cylindrical filter (240) connected to a second adaptor (210), that is configured to extend into a containment device. A first insert can comprise a connector (230) that can be connected to a cap. A filter can be configured to retain tissue fragments inside a containment device.

A second insert (300) can comprise a connector (310) that can be connected to a cap (400) and a first port (330) that can comprise a first port cap. A first port (320), a second port (110), or both a first port (320) and a second port (110) can be slip tip locks.

A first insert, a second insert and a cap can comprise a ring that contacts the one or more inner walls of the containment device to generate a closed system. A ring can be located between a connector and two adaptors or a first insert (250); between a first port and a connector of a second insert (330), and above a seal of a cap (430), respectively.

A system can comprise a device for resecting tissue fragments and fluid from a surgical site connected to a first adaptor. A device can be an endoscopic shaver, an endoscopic bone cutter, an endoscopic burr, or an endoscopic drill. A system can further comprise a device for collecting tissue fragments and fluid from a surgical site connected to the second adaptor. A device can be a suction wand.

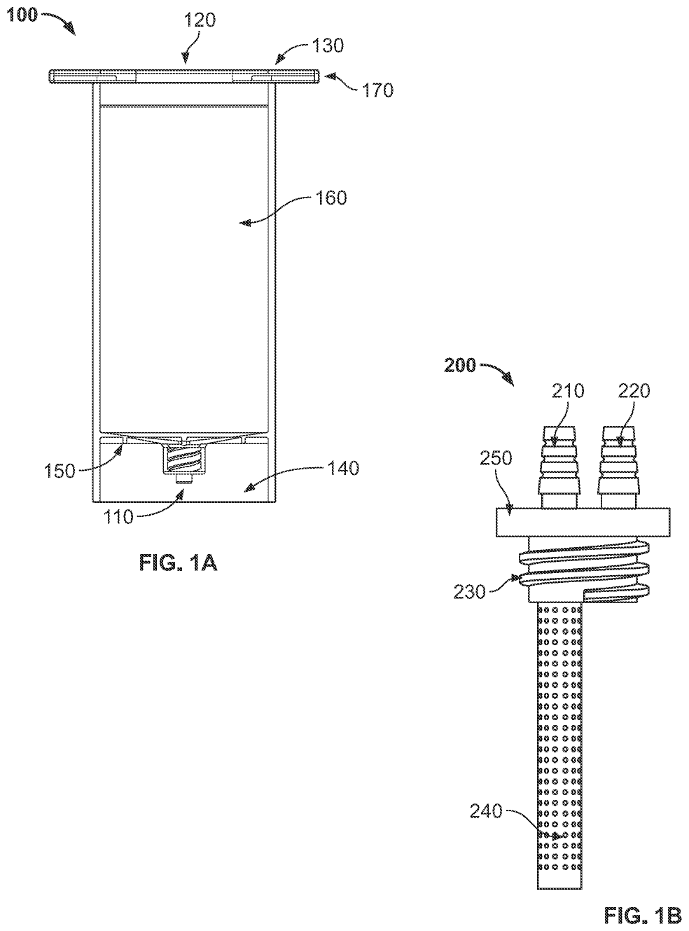

Aspects of a system for preparing a serum are exemplified herein at inter alia, FIGS. 1-3. Referring to FIG. 1, illustrating a disassembled system, a first insert (200) comprising two adaptors (210 and 220), a filter (240) and a connector (230); a second insert (300) comprising a first port (320) and a connector (310); a cap (400) comprising a seal (410) and a connector (420); and a containment device (100) are individually represented.

Containment Device

A containment device (100) can include a generally tubular body, or any other suitable shape, with an aperture (160) running from a first opening (120) at a proximal end (130), where a first insert (200), a second insert (300) and a cap (400) can be inserted, to a second opening (150) at a distal end (140), where a port (110) can be included. In some embodiments the containment device is about 2, 5, 7, 8, 9, 10, 11, 12, 13, 14, 15, 20 or more centimeters in length, and about 1, 2, 3, 4, 6, 7, 8, 9, 10, or more centimeters in diameter. A containment device can hold about 10, 20, 30, 40, 50, 60, 70, 80, 90, 100, 110, 120, 150, 200, 250, 300 or more mL of liquid/fragments.

A containment device (100) can be made of polypropylene or any other suitable solid, nonabsorbent material, and can be partially or fully translucent for visibility. For example, a containment device can be a centrifuge capable tube. A containment device can be designed to further include graduated marks for measuring a volume of tissue fragments or fluid collected in the containment device.

An aperture (160) running lengthwise of the containment device can accommodate a cap, alone or connected to either a first insert or a second insert assembled onto a cap, through a connector.

A first opening (120) at a proximal end can be a complete opening, that is the first opening can have the same size as the circumference of a containment device. Alternatively, a first opening can be a partial opening, that is the first opening can have a size that is smaller than the circumference of a containment device. In any case, a size of an insert (a first insert, a second insert or a cap) can be configured such that the ring of an insert has the same size as the circumference of the first opening of a containment device, such that an insert can fit inside or onto a containment device. A second opening (110) at a distal end can be smaller than the circumference of a containment device. For example, a second opening at a distal end can comprise a port such as a slip lip port. A slip lip port can include a port cap. Where a containment device needs to be completely sealed, a port cap can be connected to a slip lip port to avoid any material contained inside a containment device to leak from the second opening.

In one embodiment shown in FIG. 1, the containment device can comprise one or more edges (170) for easier handling of the device. For example, the device can comprise 1, 2, 3 or more edges, localized at the proximal end (130) of the device. An edge can be for example a single continuous edge, running along the entire circumference of the first opening of a device. Alternatively, a device can comprise one or more edges, that do not run along the entire circumference of the first opening of a device.

A containment device can comprise activating materials, concentrating materials, or both activating materials and concentrating materials.

An activating material can comprise a material with a solid surface or a porous surface, that can be a continuous or discontinuous material that can induce a change (e.g., an activation) of the cells present in a biological sample. For example, the activating material can comprise beads. The activating beads can be, e.g., a glass or glass-like composition such as, borosilicate glass, alumina, silicate, quartz, bioglass, ceramic glass, flint glass, fluorosilicate glass, phosphosilicate glass, and cobalt glass or conundrum. In an embodiment, an activating material can have a spherical shape to provide for a maximum surface area for contact with the biological sample. The activating material can be coated to maximize the production of, for example, IL-1Ra by monocytes within the biological fluid. The coating can be, e.g., silane, surfactants, polyether, polyester, polyurethane, or polyol groups. The activating beads or materials can range in size from about 0.1, 0.5., 1.0, 2.0, 3.0, 4.0, 5.0 mm or more. The diameter of the activating beads or activating materials can be tailored to maximize the volume of biological sample injected in the container and maximize the surface area for biological sample/bead contact. In an embodiment, an activating material comprises glass or polyacrylamide beads.

A concentrating material can be a continuous or discontinuous material that can reduce the volume of liquid a biological sample (e.g., concentrating the elements contained within the biological sample). For example, the concentrating material can comprise porous beads. The concentrating material can comprise gels, wool, powder, plastic, granules, fibers, or porous material that can absorb liquid contained the biological sample. Various examples of suitable concentration materials include glasses, minerals such as corundum and quartz, polymers, metals, and polysaccharides. Polymers include, for example, polystyrene, polyethylene, polyvinyl chloride, polypropylene, and polyacrylamide. Polysaccharides include, for example, dextran and agarose. The concentrating material can be about 0.1, 0.5, 1.0, 2.0, 3.0, 4.0, 5.0 mm or more. The diameter or size of the concentrating material can be tailored to maximize the volume of biological sample injected into the containment device and to maximize the surface area for biological sample/concentrating material contact. The diameter or size of the concentrating material can increase with the volume of liquid absorbed by the concentrating material. The concentrating material can comprise a continuous porous network, similar to a sponge, or can include a plurality of individual porous particles. The concentrating material can provide a larger surface area by being porous in comparison to a non-porous material. In some embodiments, the pores are too small for cells, e.g., blood cells, to enter, but a portion of the liquid sample can enter the pores. Liquid can be removed from the concentrating material and pores by centrifuging, for example. In some embodiments, the concentrating material can comprise a hygroscopic material, such as desiccating polyacrylamide beads, that absorb a portion of the biological sample liquid. In an embodiment, a concentrating material comprises porous glass beads or polyacrylamide beads.

A containment device can comprise one or more activating materials, one or more concentrating materials or both activating materials and concentrating materials. That is, a containment device can comprise activating materials only, for the sole purpose of activating cells present in the biological sample. A containment device can comprise concentrating materials only, for the sole purpose of reducing the volume of liquid present in the biological sample. A containment device can comprise both activating materials and concentrating materials, to simultaneously activate cells present in the biological sample and to reduce the volume of liquid present in the biological sample.

Cap

A cap (400) can be of any suitable shape. A cap can be designed to be inserted inside a containment device, and fully in contact with an inner wall or inner walls of a containment device through a seal. A cap can be designed to include a groove, running around an outer circumference of the cap that can fit a seal (410). The cap can move within the containment device (towards the proximal or distal ends) while keeping fluid from moving between the seal and the wall or walls of the containment device. In an example, a seal can be an O-ring.

An O-ring (e.g., a toric joint) can be a loop of elastomer with a round cross-section designed to be seated in a groove and compressed during assembly between two or more parts (e.g., a cap and a containment device), creating a seal at the interface. An O-ring can be made of synthetic rubber, such as butadiene rubber, butyl rubber, chlorosulfonated polyethylene, epichlorohydrin rubber, ethylene propylene diene monome, ethylene propylene rubber, fluoroelastomer, nitrile rubber, perfluoroelastomer, polyacrylate rubber, polychloroprene (neoprene), polyisoprene, polysulfide rubber, polytetrafluoroethylene, sanifluor, silicone rubber, or styrene-butadiene rubber; or thermoplastic, such as thermoplastic elastomer, thermoplastic polyolefin, thermoplastic polyurethane, polyether, polyester, thermoplastic etheresterelastomers, thermoplastic polyamide, melt processible rubber or thermoplastic vulcanizate.

A cap can be made of polypropylene or any other suitable solid, nonabsorbent material. A cap can comprise an aperture, such that a first insert or a second insert can be connected to the cap and inserted into a containment device. An aperture in a cap can be a complete opening, that is the aperture can have approximately the same size as the circumference of a cap. Alternatively, an aperture can be a partial opening, that is the aperture can have a size that is smaller than the circumference of a cap. In any case, a size of a first insert or of a second insert can be configured such that the ring of the first or second insert has the same size as the circumference of the aperture of a cap, such that a first or a second insert can fit inside or onto a cap. In some embodiments an aperture in a cap can be about 0.1, 0.2, 0.3, 0.4, 0.5, 0.6, 0.7, 0.8, 0.9, 1.0, 1.5, 2.0, 3.0, 3.5, 4.0, 5.0, 6.0 or more centimeters in diameter.

Once inserted into a containment device, a cap can define an upper chamber (e.g., the space in the containment device that is above the cap) and a lower chamber (e.g., the space in the containment device that is below the cap). An aperture in the cap can allow fluid to move from an upper chamber of the containment device to a lower chamber of the containment device or from a lower chamber of the containment device to an upper chamber of the containment device. In an example, a cap can have a ring shape.

A cap can comprise a connector that can be connected to a first insert and to a second insert, that is a first insert and a cap, or a second insert and a cap can both include a suitable connector to allow a first or a second insert to be physically attached to a cap. A connector can be, for example, screw threads, press-fit, or another suitable connector.

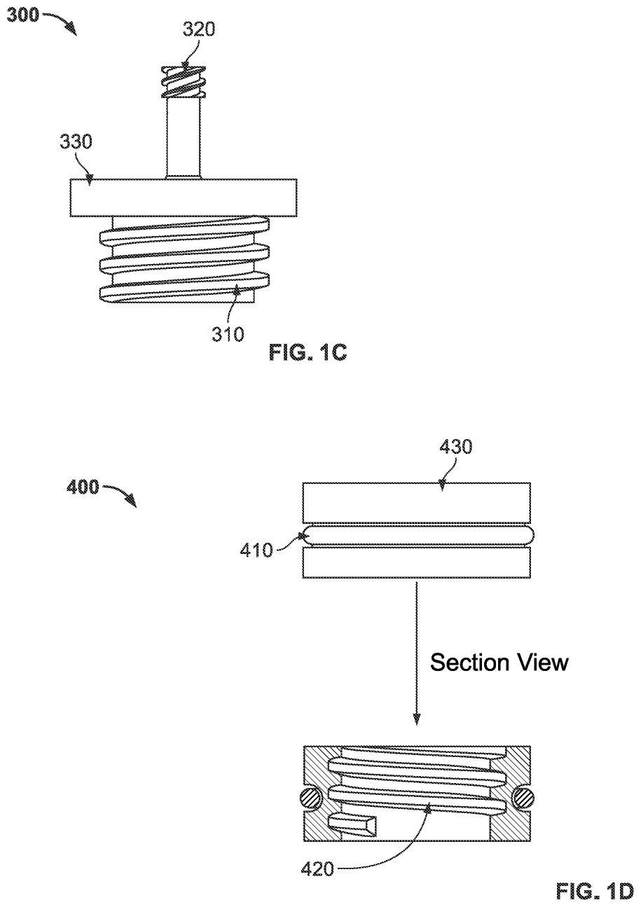

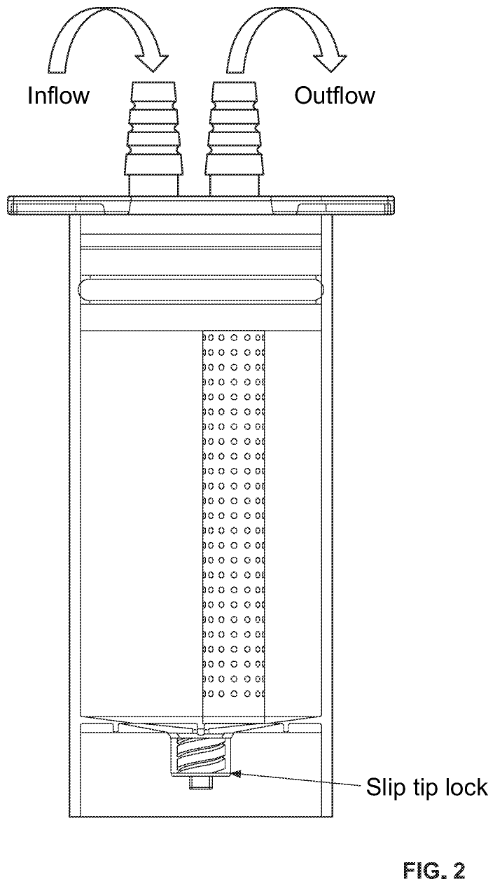

A first insert, a second insert, and a cap can each comprise screw threads, press-fit, or another suitable connector (see FIG. 1). For example, the first insert or second insert can include an internal screw thread and the cap can include an external screw thread, or alternatively, the first insert or second insert can include an external screw thread and cap can include an internal screw thread. The internal screw thread and external screw thread are designed to be complementary to one another, so that the first insert or second insert and the cap can fit together to form a sealed joint (see FIGS. 2 and 3). Alternatively, the first insert or second insert and the cap can both include a press-fit mechanism. A press-fit mechanism can be used to fasten the first insert or second insert to the cap. A press-fit mechanism, interference fit, or friction fit is a fastening between two parts (i.e., the first insert or second insert and the cap) which can be achieved by friction after the parts are pushed together, rather than by any other means of fastening, such as a screw thread for example. The pressing operation can lead to a mechanical joint between a first insert (or second insert) and a cap, leading to the assembly of both parts.

First Insert

A first insert (200) can comprise two adaptors: a first adaptor that can be configured to allow a fluid to flow into a containment device through a first insert (220) and a second adaptor that can be configured to allow a fluid to flow out of a containment device through the first insert (210). A first adaptor can be configured so that a second device for resecting tissue fragments from a surgical site, such as an endoscopic shaver, an endoscopic bone cutter, an endoscopic burr, an endoscopic drill, or the like can be connected to a device. A second adaptor can be configured so that a suction or aspiration apparatus, such as a suction wand, can be connected to a device. As used herein, the term “adaptor” refers to any universal hose or fitting, regardless of its shape. A device can include a first adaptor and a second adaptors that are universal adaptors, so that virtually any resecting device can be connected to a first adaptor, and virtually any collection device (e.g., suction or aspiration device) can be connected to a second adaptor, regardless or the brand, connection, and characteristics of the devices. A first universal adaptor and a second universal adaptor are provided for the easy assembly of the device. Any suitable adaptor can be used.

A first insert can comprise a cylindrical filter (240) connected to a second adaptor, configured to extend into a containment device.

A filter can have any shape, configured such that a filter comprises one or more side walls and a base. For example, a filter can have a circular section, and be a cup-shaped filter or a cylindrical or tubular filter having a single side wall and a circular base; a filter can have triangular section shape and have three side walls and a triangular base; a square or rectangular section shape, and have 4 side walls and a square or rectangular base. However, other shapes are possible for the filter, including for example, a hexagon or an oval.

A filter can have a length that allows a second insert to fit inside a containment device. For example, a filter can extend along the entire length of a containment device as exemplified in FIG. 2. Alternatively, a filter can extend partially into a containment device. For example, a filter can extend along 30, 40, 50, 60, 70, 80, 90, 95% or more of the length of the containment device. A bottom part of a filter can reach to a base or floor of containment device (at the distal end of the containment device); alternatively, a bottom part of a filter can be located anywhere along a length of a containment device, and not reach the base or floor of containment device. In some embodiments a filter is about 2, 3, 4, 5, 6, 7, 8, 9, 10, 12, 15, or more centimeters in length and about 0.5, 0.7, 1.0, 1.2, 1.5, 2.0, 3.0, 4.0, 5.0, 6.0, 7.0, 8.0 centimeters or more in diameter.

The base of a filter, the one or more side walls of a filter or both a base and side walls of a filter can comprise a filter material. That is the one or more sidewalls and the base can comprise a filter material, a solid material perforated, or a combination of a filter material and solid material. The solid material can include polystyrene or another solid, nonabsorbent material, and can be partially or fully translucent for visibility. The solid material can be perforated, that is comprise a large number of small holes to provide an effective flow communication between the interior of the containment device and the interior of the filter, without allowing tissue fragments to pass through the holes. The filter material can include a membrane or a mesh having pores, which can have varying size. The solid filter material can comprise for example, metals or plastics, like titanium, stainless steel, polyethylene, polytetrafluoroethylene PTFE, polyvinylidene fluoride (PVDF), nylon, polypropylene (PP), polyester, polycarbonate, polyethersulfone, cellulose acetate, polyimide, or another equivalent material. A filter can have a pore size ranging from about 50 microns to about 1000 microns. For example, a filter can have about 50 micron to about 100 micron pores, about 100 micron to about 200 micron pores, about 200 micron to about 300 micron pores, about 300 micron to about 400 micron pores, about 400 micron to about 500 micron pores, about 600 micron to about 600 micron pores, about 600 micron to about 700 micron pores, about 700 micron to about 800 micron pores, about 800 micron to about 900 micron pores, or about 900 micron to about 1000 micron pores.

A filter can be configured to retain tissue fragments inside a containment device. That is, a filter can comprise pores having a size compatible with the size of the tissue fragments liberated and collected. For example, if an assembled system is connected to a second device for resecting tissue fragments that generates tissue fragments that are greater in size than e.g., 200 microns, a filter can be designed to have about e.g., 150 micron pores, so that tissue fragments having a size greater than about 150 microns can be collected. That is, tissues fragments flowing inside a tissue containment device through a first adaptor cannot flow out of a containment device through the filter and the second adaptor and can therefore be collected inside a containment device.

A first insert can comprise a connector that can be connected to a cap. For example, a first insert can comprise a screw thread, press-fit, or another suitable connector, that is compatible with a connector of a cap.

For example, a first insert can include an internal screw thread and a cap can include an external screw thread, or alternatively, a first insert can include an external screw thread and a cap can include an internal screw thread. The internal screw thread and external screw thread are designed to be complementary to one another, so that a first insert and a cap fit together to form a sealed joint (see FIG. 2). Alternatively, a first insert and a cap can both include a press-fit mechanism. A press-fit mechanism can be used to fasten the first insert to the cap. A press-fit mechanism, interference fit, or friction fit is a fastening between two parts (i.e., a first insert and a cap) which can be achieved by friction after the parts are pushed together, rather than by any other means of fastening, such as a screw thread for example. The pressing operation can lead to a mechanical joint between the first insert and the cap, leading to the assembly of both parts.

Second Insert

A second insert (300) can comprise a connector (310) that can be connected to the cap. For example, a second insert can include an internal screw thread and a cap can include an external screw thread, or alternatively, a second insert can include an external screw thread and a cap can include an internal screw thread. The internal screw thread and external screw thread are designed to be complementary to one another, so that a first second and a cap fit together to form a sealed joint (see FIG. 3). Alternatively, a second insert and a cap can both include a press-fit mechanism. A press-fit mechanism can be used to fasten the second insert to the cap. A press-fit mechanism, interference fit, or friction fit is a fastening between two parts (i.e., a second insert and a cap) which can be achieved by friction after the parts are pushed together, rather than by any other means of fastening, such as a screw thread for example. The pressing operation can lead to a mechanical joint between a second insert and a cap, leading to the assembly of both parts.

A second insert can comprise a first port (320) that can comprise a first port cap, such as e.g., a slip tip lock. A slip lip port can include a port cap, that is if a containment device connected to a second insert needs to be completely sealed, a port cap can be connected to a slip lip port to avoid any material contained inside a containment device to leak from said second opening.

Methods of Use

Systems described herein can be used to prepare an enriched biological sample, such as enriched serum. Enriched biological samples prepared using the systems described herein can be used to treat inflammation, inflammatory joint, or rheumatoid arthritis in a subject, and to prevent inflammation of a joint following an arthroscopic surgery in a subject.

A method of producing a growth factor and/or cytokine enriched biological sample is also provided herein. For example, an interleukin-1 receptor antagonist (IL-1Ra)-enriched biological sample can be produced using methods described herein. Additionally, other cytokine- or growth factor-enriched biological samples can be produced. Examples of cytokines and growth factors include IL-1Ra, IL-6, IL-10, VEGF, EFG, HGF, PDGF, BMP-9, IGF, TGF-β, and other factors. In certain embodiments, a cytokine and/or growth factor (e.g., IL-1Ra, IL-6, IL-10, VEGF, EFG, HGF, PDGF, BMP-9, IGF, TGF-β, and other factors) enriched biological sample produced using the methods described herein comprises about 5, 10, 15, 20, 25, 30, 40 or more times the cytokines and/or growth factors as compared to a non-enriched biological sample. In an embodiment a cytokine and/or growth factor-enriched serum comprises about 100, 500, 1,000, 1,500, 2,000, 2,500% or more cytokines and/or growth factors as compared to a non-enriched biological sample.

A system can be configured such that a first insert can be connected to a cap located within a containment device at the proximal end of a containment device. A second cap be placed on a second port of a containment device to seal a containment device and avoid the leak of any fluid. A first adaptor of a first insert can be connected to a device for liberating fragments from a tissue in a fluid and a second adaptor of a first insert can be connected to a suction or aspiration apparatus to collect tissue fragments in a containment device and on a filter. A device for liberating cells can be an arthroscopic shaver, an arthroscopic bone cutter, an arthroscopic burr, or an arthroscopic drill. A suction or aspiration apparatus can be a suction wand. That is, when a suction of aspiration apparatus is turned on, and tissue fragments are liberated from a surgical site, tissue fragments can be liberated in a fluid, which can flow in the containment device through a first adaptor and through a first insert. The suction or aspiration apparatus can be in direct connection with the filter, such that any fluid that is aspirated out of the containment device must pass through a filter prior to exiting a containment device. The size of the pores of a filter is configured such that liberated tissue fragments have a greater size than the pore size, such that tissue fragments can be retained in a containment device by a filter, while fluid can freely be removed from a containment device, allowing the collection of tissue fragments inside a containment device. The aspiration/collection can last as long as needed to collect the desired amount of tissue fragments.

During the aspiration of the fluid, tissue fragments can be attracted along with fluid along the filter and remain stuck on the exterior side walls of a filter. The first insert can be removed or disconnected from the cap, and tissue fragments can be removed from the filter to pool tissue fragments in a containment device. Removing tissue fragments from a filter can comprise scraping tissue fragments from a filter and into the containment device. For example, tissue fragments can be dissociated from the filter by scraping a filter on the sides of a containment device while removing a first insert. Alternatively, tissue fragments can be dissociated from a filter using, e.g., a lab spoon or spatula or backflushing with fluid.

A first insert can be removed, and a second insert can then be connected to the cap. The first port (320) of the second insert (300) can comprise a first cap to seal the system. Some fluid can remain in the containment device at the end of an aspiration step. A system comprising a containment device and a second cap, a cap, a second insert and a first cap can then be centrifuged to separate tissue fragments from any remaining fluid. A containment device can be centrifuged for about 5, 10, 20, 30, 40 or more minutes. A containment device can be centrifuged at a speed of about 200, 300, 400 500, 750, 1000, 1500, 2000, 2500, 3000, 4000×g or more.

After centrifugation, tissue fragments can be pooled at a distal end of the containment device (by the second port (110)) with any remaining fluid above. The first cap of a second insert can be removed, and the fluid can be withdrawn from a containment device using a first port of a second insert. Removing the fluid can comprise, for example, connecting a syringe to a first port of a second insert and collecting the fluid in the syringe. As illustrated in FIG. 3, collecting the fluid from a containment device, e.g., in a syringe, can actuate the cap and a second insert down within a containment device toward the distal end of a containment device. Alternatively, after removing a first cap of a second insert, a second insert and a cap to which it is connected can be manually actuated downwards in a containment device such that the fluid can be evacuated through a first port. The fluid can then sit on top of the cap/second insert assembly, and be evacuated by decanting the system, or by aspirating it (after optionally placing the first cap back on the first port of the second insert to avoid any fluid entering back into the containment device). When removing the fluid by decanting or aspiring the fluid from the top of the cap/second insert assembly, depending on the volume of fluid to be removed, the removal can be done in several repetitive steps where the first cap is alternatively removed to actuate the assembly down, placed back to avoid overflow of fluid back in the containment device, and removed to repeat the operation.

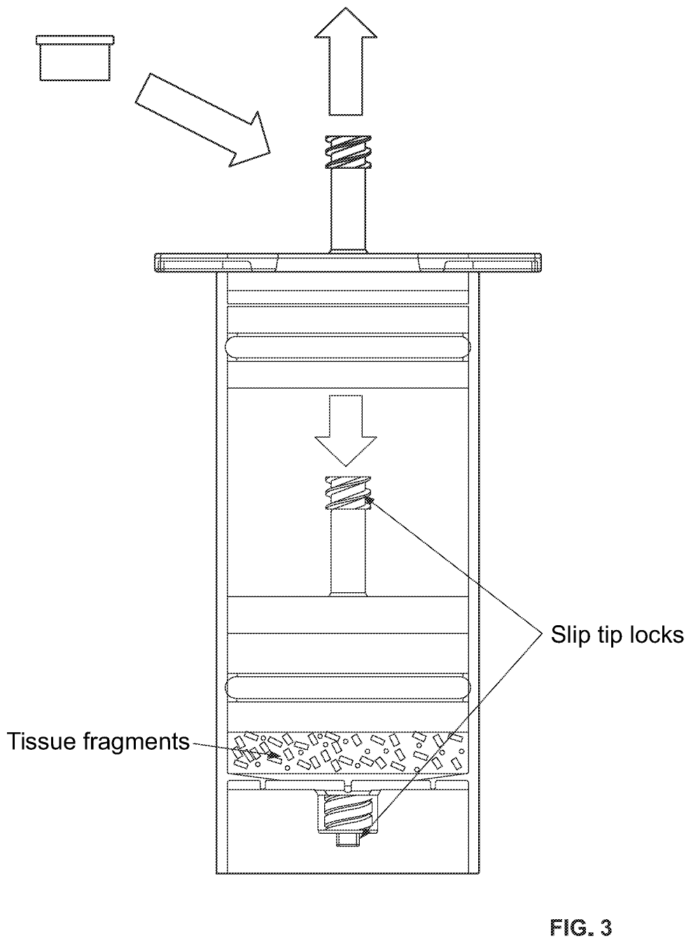

As illustrated in FIG. 4, once tissue fragments are isolated from any remaining fluid, a biological sample can be injected into a containment device. A biological sample can be injected into a containment device using a first port of a second insert or in a second port of a containment device. To avoid any leakage of biological sample throughout the device, a first cap or a second cap can be placed on a first port or on a second port, while the biological sample is injected into the uncapped port. For example, a first cap can be placed on a first port of the second insert, and a biological sample can be injected into a containment device via a second port of a containment device. In another example, a second cap can be placed on a second port of the containment device, and a biological sample can be injected in a containment device via a first port of a second insert. Injecting the biological sample in a containment device with a syringe can actuate the cap/second insert assembly up within a containment device toward the proximal end of a containment device. Alternatively, prior to the injection of the biological sample in a containment device, the cap/second insert assembly can be manually actuated upwards.

A biological sample can be whole blood, serum, buffy coat, plasma, platelet rich plasma, bone marrow, effusion fluid, synovial fluid, an adipose sample such as a processed adipose sample, or any other suitable sample. A processed adipose sample can be harvested adipose tissue that is, for example, centrifuged and washed or processed mechanically to break up the adipose cells into smaller fragments, ridding it of blood and other potential inflammatory sources. A biological sample can be any biological fluid that can comprise cells that can produce, or be activated to produce compounds of interest, e.g., cytokines or growth factors IL-1Ra, IL-6, IL-10, VEGF, EFG, HGF, PDGF, BMP-9, IGF, TGF-β, and other factors. For example, a biological sample can be a biological fluid that can comprise monocytes.

A biological sample can then be incubated with tissue fragments. In some embodiments, the biological sample is incubated at room temperature or at about 5, 10, 20, 30° C. or more. The biological sample can be incubated for 15 or more minutes or one or more hours, for example, the biological sample can be incubated with the tissue fragments for about 15, 30, 45 or more minutes or about 1, 2, 3, 4, 5, 6, or more hours. In an exemplary embodiment, the biological sample and the tissue fragments are incubated for about 4 hours. Optionally, the tissue fragments can be homogenized or mixed in the biological sample. Homogenization or mixing of the tissue fragments and biological sample can be performed one or more times during the incubation period. For example, homogenization or mixing can be performed 1, 2, 3, 4 or more times during the incubation period. For example, the biological sample and tissue fragments can be homogenized or mixed every other hour, every hour, every half-hour or every 10 minutes to avoid sedimentation of the tissue fragments, and to ensure maximal contact of the tissue fragments with the blood sample. Homogenization or mixing can be done by manually inverting the system one or more times during the incubation period. Alternatively, the system can be installed on a device to allow for the continuous homogenization or mixing of the tissue fragments with the biological sample during the incubation period. For example, the system can be placed on an orbital shaker, a rocker, a tube revolver, a rotating shaker, and the like.

The containment device can comprise one or more activating materials, one or more concentrating materials, or both activating materials and concentrating materials. That is, activating, concentrating, or both activating and concentrating materials can be added to the containment device such that they are in contact with the biological sample and the tissue fragments.

Activating, concentrating, or both activating and concentrating materials can be added to the containment device at various time points including prior to introduction of tissue fragments or after collection of tissue fragments in the containment device. For example, activating, concentrating, or both activating and concentrating materials can be added to the tissue fragments after centrifugation and removal of the fluid (e.g., activating, concentrating, or both activating and concentrating materials can be added to the tissue fragments prior to the injection of the biological sample). Activating, concentrating, or both activating and concentrating materials can also be added to the containment device after injection of the biological sample (e.g., activating, concentrating, or both activating and concentrating materials can be added to the tissue fragments and biological sample prior to the incubation period).

In another example, activating, and concentrating materials can be added to the tissue fragments and biological sample at different times. For example, activating materials can be added to the tissue fragments after centrifugation and removal of the fluid but prior to the injection of the biological sample; or after injection of the biological sample but prior to the incubation period. Concentrating materials can be added to the tissue fragments after centrifugation and removal of the fluid but prior to the injection of the biological sample; after injection of the biological sample but prior to the incubation period; or after incubation of the tissue fragments with the biological sample (with or without activating materials).

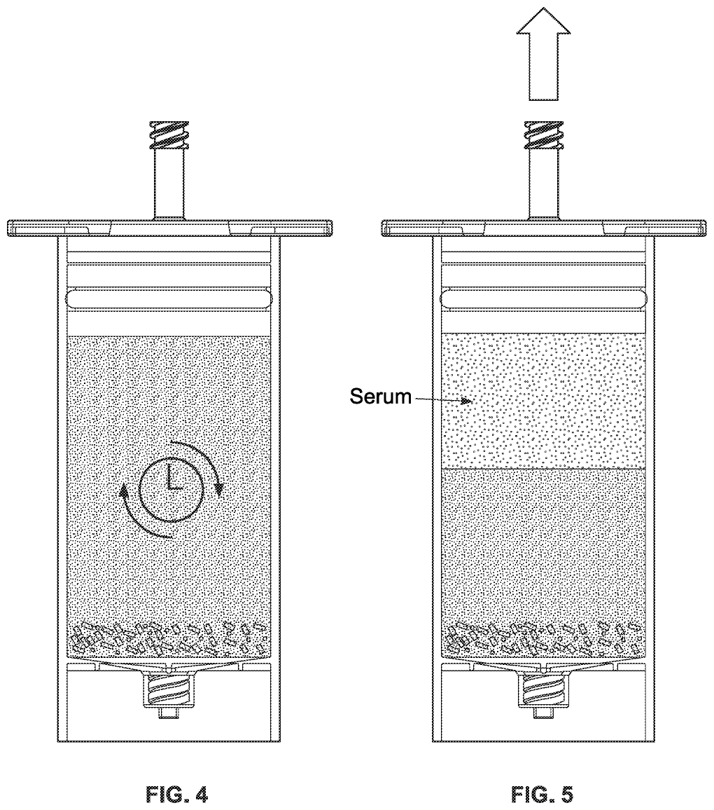

After the incubation period, a system, comprising a containment device and a second cap, a cap, a second insert and a first cap can then be centrifuged to separate an enriched biological sample from the biological sample (e.g., non-enriched biological sample), and to precipitate the tissue fragments and, for example, red blood cells or other undesirable components of the non-enriched biological sample (as illustrated in FIG. 5, the serum fraction is the enriched biological sample in this example). A containment device can be centrifuged for about 5, 10, 20, 30, 40, 50, 60 or more minutes. A containment device can be centrifuged at a speed of about 1000, 2000, 3000, 4000, 5000 or more rpm. For example, a containment device can be centrifuged for 10 minutes at 4000 rpm. In one embodiment, the biological sample is a blood sample, and the enriched biological sample is an enriched serum. The method can comprise collecting a serum, e.g., a cytokine or growth factor enriched serum, such as an IL-1Ra-enriched serum, from a containment device.

After centrifugation, tissues fragments can be pooled at a distal end of a containment device (by a second port) with the biological sample separated into a dense fraction (located at a distal end of the containment device), and a light fraction e.g., the enriched biological sample (located above the dense fraction. For example, a biological sample can be a blood sample (such as a non-anticoagulated blood sample) separated into a serum fraction and a red blood cell (RBC) fraction. A RBC fraction can be more dense than a serum fraction, and can be located at the distal end of the containment device, while a serum fraction can be less dense than a RBC fraction, and be located above a RBC fraction. A first cap of a second insert can be removed, and an enriched serum can be withdrawn from a containment device using a first port of a second insert.

Removing the fluid can comprise, for example, connecting a syringe to a first port of a second insert and collecting the fluid in a syringe. Collecting the fluid from a containment device, e.g., in a syringe, can actuate a cap and a second insert down within a containment device toward the distal end of a containment device. Collecting a cytokine and/or growth factor enriched serum (e.g., IL-1Ra-enriched serum) can comprise connecting a syringe to a first port of a second insert and collecting the IL-1Ra-enriched serum in a syringe.

A cytokine or growth factor enriched serum (e.g., IL-1Ra IL-6, IL-10, VEGF, EFG, HGF, PDGF, BMP-9, IGF, and/or TGF-β enriched serum) can comprise increased cytokine and/or growth factor levels as compared to a non-enriched serum. For example, a growth factor and/or cytokine-enriched serum can comprise about 1000, 2000, 3000, 4000, 5000, 6000, 7000 or more pg/ml of each growth factor or cytokine. In an embodiment, a growth factor and/or cytokine-enriched serum comprises about 5, 10, 15, 20, 25, 30, 40 or more times the cytokine or growth factor as compared to a non-enriched serum. In an embodiment a growth factor and/or cytokine-enriched serum comprises about 100, 500, 1,000, 1,500, 2,000, 2,500% or more growth factors or cytokines as compared to a non-enriched serum.

A method of treating inflammation, inflammatory joint, or rheumatoid arthritis in a subject is provided. A method of preventing inflammation of a joint following an arthroscopic surgery in a subject is also provided. The methods described herein can comprise administering to the subject a cytokine and/or cytokine-enriched biological sample, e.g., an IL-1Ra-enriched biological sample, produced by the methods described herein. The administration of a cytokine and/or cytokine-enriched biological sample, e.g., an IL-1Ra-enriched biological sample, can treat the inflammation or relieve the symptoms associated with it. A cytokine and/or cytokine-enriched biological sample, can be, e.g., an IL-1Ra-enriched blood sample such as an IL-1Ra-enriched serum.

By “administering a cytokine and/or growth factor enriched serum”, it is meant that a cytokine and/or growth factor-enriched serum (e.g., an IL-1Ra enriched serum) prepared as described herein can be injected to a subject in need thereof (e.g., a subject suffering from or at risk of developing inflammation, inflammatory joint, or rheumatoid arthritis). The cytokine and/or growth factor-enriched blood sample can be injected at an inflammation site, inflammatory joint, or rheumatoid arthritis site. For example, a cytokine and/or growth factor-enriched serum can be injected into a knee joint, shoulder joint, ankle joint, elbow joint, hip joint, or wrist joint.

As used herein, the term “inflammation” can refer to a process induced by white blood cells to protect from infection. Some diseases, such as arthritis, can trigger an inflammation response even in the absence of an infection-causing pathogen. In such cases, e.g., when the subject has an autoimmune disease, the immune system acts as if regular tissues were infected which can cause tissue damage. Inflammation can be acute (short-lived, e.g., goes away within hours or days) or chronic (long-lasting, e.g., can last months or years, even after the first trigger is gone). Conditions linked to chronic inflammation can include cancer, heart disease, diabetes, asthma, Alzheimer's disease, and arthritis, such as rheumatoid arthritis, psoriatic arthritis, or gouty arthritis. Other painful conditions of the joints and musculoskeletal system that may or may not be related to inflammation include osteoarthritis, fibromyalgia, muscular low back pain, and muscular neck pain.

“Inflammatory joint” or “arthritis” can be used to describe conditions characterized by pain, swelling, tenderness and warmth in the joints, as well as morning stiffness that lasts for more than an hour. The most common forms are rheumatoid arthritis (RA), psoriatic arthritis (PsA), systemic lupus erythematosus (SLE, lupus), gout and ankylosing spondylitis (AS). In these diseases, the immune system doesn't work properly and releases inflammatory chemicals. The resulting inflammation attacks joint tissues and can cause joint swelling, increased joint fluid, cartilage and bone damage, and muscle loss. Nerves in the joints are also activated, causing pain. The inflammatory chemicals can directly activate other nerves of the body and lead to pain as well.

The term “subject” as used herein refers to any individual or patient to which the subject methods are performed. Generally, the subject is human, although as will be appreciated by those in the art, the subject can be an animal such as a mammal. Thus other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject. For example, the subject can be a mammal including but not limited to a human, equine, canine, feline, bovine, porcine, rodent, sheep, or goat.

The term “treatment” is used interchangeably herein with the term “therapeutic method” and refers to both 1) therapeutic treatments or measures that cure, slow down, lessen symptoms of, and/or halt progression of a diagnosed pathologic conditions or disorder, and 2) and prophylactic/preventative measures. Those in need of treatment can include individuals already having a particular medical disorder as well as those who can ultimately acquire the disorder (i.e., those needing preventive measures).

The terms “administration of” and or “administering” should be understood to mean providing a pharmaceutical composition in a therapeutically effective amount to the subject in need of treatment. As such, administration routes of the cytokine and/or growth factor enriched biological sample (e.g., an IL-1Ra-enriched biological sample) described herein can include but are not limited to intracutaneous, subcutaneous, intracapsular, intraarticular, subcapsular, and intraspinal administrations. In one embodiment, a cytokine and/or growth factor enriched sample, e.g., an IL-1Ra-enriched biological sample can be injected at an inflammation site, inflammatory joint, or rheumatoid arthritis site.

In some aspects, administration can be in combination with one or more additional therapeutic agents. The phrases “combination therapy”, “combined with” and the like refer to the use of more than one medication or treatment simultaneously to increase the response. The composition of the present invention can for example be used in combination with other drugs or treatment in use to treat inflammation. Specifically, the administration of the cytokine and/or growth factor enriched sample (e.g., a IL-1Ra-enriched biological sample) to a subject can be in combination with an anti-inflammatory molecule. Such therapies can be administered prior to, simultaneously with, or following administration of the composition of the present invention.

The phrase “anti-inflammatory molecule” refers to any molecule capable of inhibiting or reducing an inflammatory response. Examples of anti-inflammatory molecules include, but are not limited to, IL-1 receptor antagonist, IL-4, IL-6, IL-10, IL-11, IL-13, nonsteroidal anti-inflammatory drugs (NSAIDs, such as aspirin, ibuprofen, or naproxen), corticosteroids, calcineurin inhibitors, TGF-β, vitamin D and retinoic acid.

Corticosteroids are a class of steroid hormones that are produced in the adrenal cortex of vertebrates, as well as the synthetic analogues of these hormones. Two main classes of corticosteroids, glucocorticoids and mineralocorticoids, are involved in a wide range of physiologic processes, including stress response, immune response, and regulation of inflammation, carbohydrate metabolism, protein catabolism, blood electrolyte levels, and behavior. Some common naturally occurring steroid hormones are cortisol, corticosterone, and cortisone. Other examples of corticosteroids include prednisone, prednisolone, dexamethasone, budesonide, beclomethasone dipropionate, triamcinolone acetonide, fluticasone propionate, fluticasone furoate, flunisolide, methylprendisone and hydrocortisone.

Calcineurin inhibitors suppress the immune system by preventing interleukin-2 (IL-2) production in T cells. Examples of calcineurin inhibitors include cyclosporine and tacrolimus. Cyclosporine and tacrolimus bind to the intracellular immunophilins cyclophilin and FKBP-12, respectively. When bound, both molecules inhibit the phosphatase action of calcineurin, which is required for the movement of nuclear factors in activated T cells to the chromosomes where subsequent cytokine synthesis occurs. Decreased secretion of IL-2 prevents proliferation of the inflammatory response via B cells and T cells. The attenuated inflammatory response greatly reduces the overall function of the immune system.

### Definitions

As used herein, a “fluid” is a liquid collected from surgical site into a subject. The subject can be a mammal. The fluid can be an isotonic solution, such as a saline solution or Ringer's lactate solution, which is commonly used to safely provide for surgical site irrigation during surgery. The fluid can include biological fluid, collected from the subject; it can be autogenic, allogenic, or xenogenic. Biological fluids include, but are not limited to, whole blood, plasma, serum, urine, saliva, mucus, cerebrospinal fluid, lymphatic fluid, seminal fluid, amniotic fluid, vitreous fluid, as well as fluid collected from cell culture of patient cells, and the like. Biological fluids also include fluids derived from tissue such as, for example, bone, bone marrow, muscle, brain, heart, liver, lung, stomach, small intestine, large intestine, colon, uterus ovary, testis, cartilage, soft tissue, skin, subcutaneous tissue, breast tissue, tissue obtained from other species, patient tissue from surgery, and the like. Biological fluids also can include, for example, bone marrow, fluids obtained from surgery, fluid filtrates, and the like.

As used herein, the term “tissue fragment” can refer to fragments, pieces, or debris obtained from a tissue during surgery. The tissue can be, e.g., soft tissue, bone or cartilage; and the tissue fragment can include bone or cartilage filtrates or fragments, bone/cartilage chips or fragments, or any other type of tissue pieces that can result from the treatment provided at the surgical site, obtained during surgery. The soft tissue can be tendon, ligament, muscle, adipose, or fascia. The surgical site can be a joint, such as a knee, a shoulder, an ankle, an elbow, a hip, or a wrist. The tissue fragments can be collected for autologous graft.

As used herein, a “biological sample” can refer to any biological fluid that can comprise cells that can produce or can be activated to produce compounds of interest cytokines, and/or growth factors, e.g., IL-1Ra, IL-6, IL-10, VEGF, EFG, HGF, PDGF, BMP-9, IGF, TGF-β, and other factors. For example, a biological sample can be a biological fluid that can comprise monocytes. Non-limiting examples of biological samples that can be used in the methods described herein include blood, bone marrow, effusion fluid, synovial fluid, or adipose tissue, e.g., a processed adipose sample.

As used herein, a “blood sample” can refer to a sample that is usually extracted from a vein in the arm of a subject using a hypodermic needle, or via fingerprick. A blood sample can contain a representative number of the major cells in a subject's blood, such as white blood cells, red blood cells, and platelets. A blood sample can be a whole blood sample, plasma, buffy coat, serum, or combinations thereof. The blood sample can be a non-anticoagulated blood sample, a platelet-rich plasma (PRP) sample, or another concentrated blood fraction. PRP is a composition comprising an increased concentration of autologous or heterologous platelets suspended in a small amount of plasma.

The compositions and methods are more particularly described below, and the Examples set forth herein are intended as illustrative only, as numerous modifications and variations therein will be apparent to those skilled in the art. As used in the description herein and throughout the claims that follow, the meaning of “a”, “an”, and “the” includes plural reference unless the context clearly dictates otherwise. The term “about” in association with a numerical value means that the value varies up or down by 5%. For example, for a value of about 100, means 95 to 105 (or any value between 95 and 105).

The terms used in the specification generally have their ordinary meanings in the art, within the context of the compositions and methods described herein, and in the specific context where each term is used.

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising”, “consisting essentially of”, and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the embodiments claimed. Thus, it should be understood that although the present compositions and methods have been specifically disclosed by embodiments, optional features, modification and variation of the concepts herein disclosed can be resorted to by those skilled in the art, and that such modifications and variations are considered to be within the scope of these embodiments as defined by the description and the appended claims.

Any single term, single element, single phrase, group of terms, group of phrases, or group of elements described herein can be each be specifically excluded from the claims.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods

In addition, where features or aspects of the embodiments are described in terms of Markush groups or other grouping of alternatives, those skilled in the art will recognize that the embodiments are also thereby described in terms of any individual member or subgroup of members of the Markush group or other group.

The following are provided for exemplification purposes only and are not intended to limit the scope of the invention described in broad terms above.

### EXAMPLES

### Example 1

### Preparation of an Il-1Ra Enriched Serum

Tissue Collection

Tissue fragments were collected from a cadaveric knee using the tissue collector device. Predominantly cartilage was resected and collected in two containment devices and predominantly bone was resected in two additional containment devices. A first insert was connected to a cap located within a first containment device. A cap was placed on the port of the second containment device to seal a containment device and avoid the leak of any fluid. A first adaptor of a first insert was connected to a device for liberating fragments from a knee in a fluid and a second adaptor of a first insert was connected to a suction or aspiration apparatus to collect bone fragments in the first containment device and on a filter. A first insert was connected to a cap located within a second containment device. A cap was placed on the port of the second containment device to seal a containment device and avoid the leak of any fluid. A first adaptor of a first insert was connected to a device for liberating fragments from a knee in a fluid and a second adaptor of a first insert was connected to a suction or aspiration apparatus to collect cartilage fragments in the second containment device and on a filter. The extra arthroscopic fluid was removed from the containment devices until about 10 ml of tissue and fluid remained (about a 50:50 combo of tissue and saline).

The first inserts were removed or disconnected from the caps, and tissue fragments were removed from the filters to pool tissue fragments in the two containment devices. The first inserts were removed, and second inserts were connected to the caps. The port of the second inserts were capped to seal the systems. The containment devices were centrifuged, and the fluid removed.

Blood Incubation

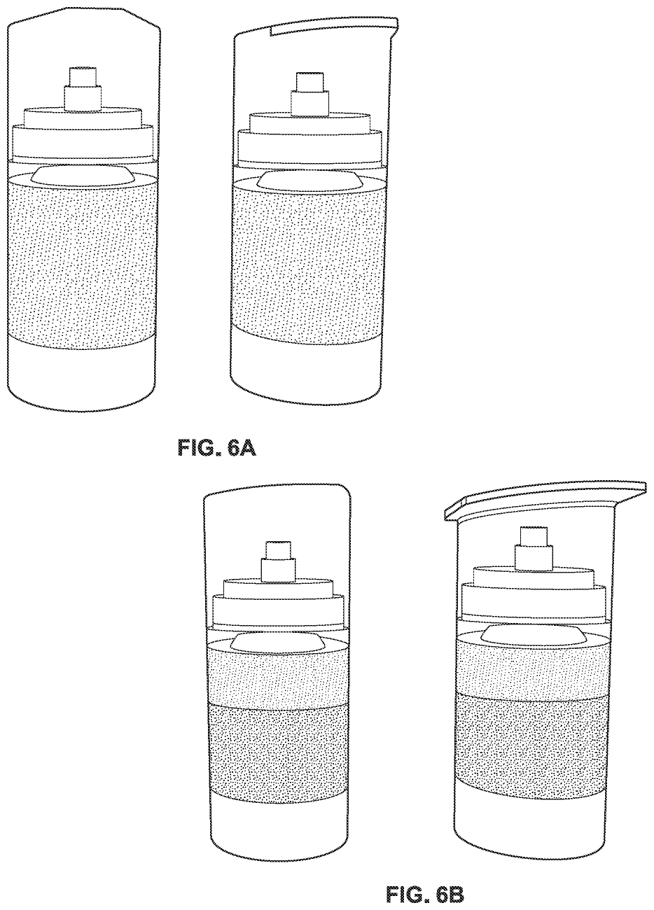

After centrifugation, the cap of the second inserts were removed, and fifty (50) ml of whole blood from two donors was injected into each containment device. The blood samples were then incubated with the tissue fragments for 4 hours (FIG. 6A). Additionally, a sample of blood was taken from each donor, spun down, and plasma extracted as a control.

After incubation, the containment devices were spun in a centrifuge for 10 minutes at 4000 rpm to precipitate the tissue fragments and separate out an RBC fraction from a serum fraction comprising the enriched serum of interest e.g., IL-1Ra-enriched serum (FIG. 6B).

The cap of the second inserts were removed, and the enriched serums were withdrawn from the containment devices using the first port of the second inserts and a syringe. Enriched serums were collected and analyzed for their content in IL-1Ra.

ELISA Analysis

An IL-1Ra enzyme-linked immunosorbent assay (ELISA) was conducted on each of the samples. IL-1Ra levels increased in experimental bone and cartilage conditions as compared to control (Table 1).

TABLE 1
Results-IL-1Ra (pg/ml)

| | Donor 1 | Donor 2 | Average |
|---|---|---|---|
| Control | 371.1 | 184.9 | 278.0 |
| Bone | 4286.3 | 5084.6 | 4685.5 |
| Cartilage | 4092.8 | 5129.3 | 4611.1 |

Although the different non-limiting embodiments are illustrated as having specific components, the embodiments of this disclosure are not limited to those particular combinations. It is possible to use some of the components or features from any of the non-limiting embodiments in combination with features or components from any of the other non-limiting embodiments. Indeed, the embodiments, examples and alternatives of the preceding paragraphs, the claims, or the following description and drawings, including any of their various aspects or respective individual features, can be practiced independently or in any combination. Features described in connection with one embodiment are applicable to all embodiments, unless such features are incompatible.

It should be understood that like reference numerals identify corresponding or similar elements throughout the several drawings. It should also be understood that although a particular component arrangement is disclosed and illustrated in these exemplary embodiments, other arrangements could also benefit from the teachings of this disclosure.

The foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

We claim:

1. A system for preparing a serum comprising:
a) a containment device (100);
b) a cap (400) that fits within the containment device comprising a seal (410), an aperture, and a connector (420) that can be connected to a first insert and to a second insert;
c) a first insert (200) comprising:
i) a first adaptor (220) configured to allow a fluid to flow into the containment device through the first insert;
ii) a second adaptor (210) configured to allow a fluid to flow out of the containment device through the first insert;
iii) a cylindrical filter (240) connected to the second adaptor, configured to extend into the containment device; and
iv) a connector (230) that can be connected to the cap;
d) a second insert (300) comprising:
i) a connector (310) that can be connected to the cap, and
ii) a first port (320) that can comprise a first port cap.

2. The system of claim 1, wherein the containment device comprises a first opening at a proximal end to accommodate the cap, and a second opening at a distal end comprising a second port that can comprise a second port cap.

3. (canceled)

4. (canceled)

5. (canceled)

6. The system of claim 1, further comprising a device for resecting tissue fragments and fluid from a surgical site connected to the first adaptor.

7. (canceled)

8. The system of claim 1, further comprising a device for collecting tissue fragments and fluid from a surgical site connected to the second adaptor.

9. (canceled)

10. (canceled)

11. (canceled)

12. (canceled)

13. The system of claim 1, wherein the containment device comprises an activating material, a concentrating material, or both an activating material and a concentrating material.

14. The system of claim 13, wherein the activating material, the concentrating material, or both the activating material and concentrating material comprise glass beads or polyacrylamide beads.

15. A method of producing a cytokine-enriched biological sample, a growth factor-enriched biological sample, or both, with the system of claim 1 comprising:
a) connecting the first insert to the cap, wherein the cap is located within the containment device at the proximal end of the containment device;
b) connecting the first adaptor of the first insert to a device for liberating fragments from a tissue in a fluid and connecting the second adaptor of the first insert to a suction or aspiration apparatus to collect tissue fragments in the containment device and on the filter;
c) removing tissue fragments from the filter to pool tissue fragments in the containment device;
d) removing the first insert and connecting the second insert of the system to the cap;
e) centrifuging the system to separate the tissue fragments from the fluid and removing the fluid from the containment device; and
f) adding a biological sample to the containment device and incubating the tissue fragments with the biological sample, thereby producing a cytokine-enriched biological sample, a growth factor-enriched biological sample, or both.

16. A method of producing an interleukin-1 receptor antagonist (IL-1Ra)-enriched biological sample with the system of claim 1 comprising:
a) connecting the first insert to the cap, wherein the cap is located within the containment device at the proximal end of the containment device;
b) connecting the first adaptor of the first insert to a device for liberating fragments from a tissue in a fluid and connecting the second adaptor of the first insert to a suction or aspiration apparatus to collect tissue fragments in the containment device and on the filter;
c) removing tissue fragments from the filter to pool tissue fragments in the containment device;
d) removing the first insert and connecting the second insert of the system to the cap;
e) centrifuging the system to separate the tissue fragments from the fluid and removing the fluid from the containment device; and
f) adding a biological sample to the containment device and incubating the tissue fragments with the biological sample, thereby producing an IL-1Ra-enriched biological sample.

17. The method of claim 15, wherein the containment device comprises an activating material, a concentrating material, or both an activating material and a concentrating material.

18. The method of claim 17, wherein the activating material, concentrating material, or both activating material and concentrating material comprise glass beads or polyacrylamide beads.

19. (canceled)

20. (canceled)

21. The method of claim 15, further comprising centrifuging the system to separate the enriched biological sample from the biological sample.

22. The method of claim 15, wherein the biological sample is a blood, bone marrow, effusion fluid, synovial fluid, or adipose tissue.

23. (canceled)

24. The method of claim 15, further comprising collecting the cytokine-enriched biological sample or the growth factor-enriched biological sample, wherein collecting the cytokine-enriched biological sample or growth factor-enriched biological sample comprises connecting a syringe to the first port of the second insert and collecting the cytokine-enriched biological sample or a growth factor-enriched biological sample in the syringe.

25. The method of claim 15, wherein collecting the cytokine-enriched biological sample or growth factor-enriched biological sample, from the containment device in the syringe actuates the cap down within the containment device toward the distal end of the containment device.

26. (canceled)

27. (canceled)

28. (canceled)

29. (canceled)

30. (canceled)

31. (canceled)

32. The method of claim 22, wherein the blood is a non-anticoagulated blood sample or a platelet-rich plasma (PRP) sample.

33. (canceled)

34. The method of claim 15, wherein the cytokine-enriched biological sample, the growth factor-enriched biological sample, or both are enriched with one or more of IL-1Ra, IL-6, IL-10, VEGF, EFG, HGF, PDGF, BMP-9, IGF, and TGF-β.

35. A method of treating inflammation, inflammatory joint, or rheumatoid arthritis in a subject comprising administering to the subject a cytokine-enriched biological sample, a growth factor-enriched biological sample, or both produced by the method of claim 15.

36. The method of claim 35, wherein the cytokine-enriched biological sample, a growth factor-enriched biological sample, or both is injected at an inflammation site, inflammatory joint, or rheumatoid arthritis site.

37. (canceled)

38. (canceled)

39. A method of preventing inflammation of a joint following an arthroscopic surgery in a subject comprising administering into the joint a cytokine-enriched biological sample, a growth factor-enriched biological sample, or both produced by the method of claim 15.

40. The method of claim 39, wherein the joint is a knee, an elbow, a shoulder, a wrist, an ankle, or a hip.

41. (canceled)
